(19) United States
(12) Patent Application Publication
(10) Pub. No.: US 2006/0106368 A1
(43) Pub. Date: May 18, 2006

(54) Smart Surgical Device Operating System With Radio Frequency Identification

(75) Inventors: Ronald H. Miller, Dearborn, MI (US); Hanaan Elmessiri, Inkster, MI (US); Scott A. Dulchavsky, Grosse Point Park, MI (US); Ilan S. Rubinfeld, Ann Arbor, MI (US); Mark A. Kelley, Bloomfield Hills, MI (US)

Correspondence Address: ARTZ & ARTZ, P.C., 28333 TELEGRAPH RD., SUITE 250, SOUTHFIELD, MI, 48034, US

(73) Assignee: SMART TECHNOLOGIES, LLC, Dearborn, MI (US)

(21) Appl. No.: 10/904,530

(22) Filed: Nov. 15, 2004

(51) Int. Cl. A61B 17/00

(52) U.S. Cl. 606/1

(57) ABSTRACT

A smart surgical device (10) for use during an operating procedure includes a surgical apparatus (12) that aids in the facilitation of an operating procedure. A body internal readable control circuit (14) is coupled and stores identification information related to the surgical apparatus (12).

### TECHNICAL FIELD

The present invention relates generally to systems utilized and techniques performed during a surgery. More particularly, the present invention relates to a system and method of detecting and accounting for surgical apparatuses during and upon completion of a surgery.

### BACKGROUND OF THE INVENTION

Surgical fluid absorbing apparatuses as well as other surgical apparatuses are used throughout an operation on a patient to aid in the facilitation of an operating procedure. Fluid absorbing apparatuses are utilized to absorb various fluids within the patient. There are various types of surgical fluid absorbing apparatuses, which can be positioned under organs deep inside a body cavity, depending upon the nature of the operation. It can be difficult to locate the various absorbing apparatuses upon completion of an operation. Difficulty in detecting the absorbing apparatuses is dependent upon the absorbing apparatus type, the location of use, and other issues associated with the operation. It is desirable to account for and remove all fluid absorbing apparatuses from within a patient upon completion of an operation.

Currently, to account for all of the absorbing apparatuses used during an operation, as each absorbing apparatus pack is opened, a nursing staff manually counts the absorbing apparatuses and hands them individually to a surgeon. As the absorbing apparatuses are used, disposed of, and new absorbing apparatuses are placed inside a body, the nursing staff tracks the total number of absorbing apparatuses used. At the end of an operation, the number of used absorbing apparatuses is compared to the number of absorbing apparatuses provided to the surgeon. When there is not a one-to-one correlation in the number used and the number of removed fluid absorbing apparatuses, time is spent to account for this discrepancy and search for the missing fluid absorbing apparatuses.

Another technique performed to account for the fluid absorbing apparatuses utilized during an operating procedure includes the use of a plastic binning process whereby five used absorbing apparatuses at a time are placed in compartmentalized plastic slots, similar to the slots of a shoe garment bag. The absorbing apparatuses are then counted and an absorbing apparatus status is determined.

A third technique that has also been performed to account for absorbing apparatuses is the use of x-ray detection. When there is a discrepancy between the number of used absorbing apparatuses and the number of removed absorbing apparatuses, surgeons may physically search the body cavity and when necessary perform an x-ray on the patient to detect the missing fluid absorbing apparatuses. Current fluid absorbing apparatuses have a radio opaque strip for identification, which is detectable and thus locatable in an x-ray.

The above-stated techniques may all be utilized during an operation. However, the locating and accounting for of absorbing apparatuses, as well as other surgical apparatuses, can be timely and costly. In general, every minute in an operating room is costly. Thus, there exists a need for an improved technique of accounting for surgical apparatuses used during an operating procedure.

### SUMMARY OF THE INVENTION

The present invention provides a smart surgical device for use during an operating procedure. The smart surgical device includes a surgical apparatus that aids in the facilitation of an operating procedure. A body internal readable control circuit is coupled and stores identification information related to the surgical apparatus.

The embodiments of the present invention provide several advantages. One such advantage is the provision of a smart surgical device, which allows for the quick and easy detection of surgical apparatuses within a body. The use of a smart surgical device also allows for the performance of an automated accounting of surgical apparatuses utilized during an operating procedure.

Another advantage provided by an embodiment of the present invention is the provision of a surgical device detection system for the detection of surgical apparatuses within a body. The system allows for the communication and/or detection of surgical devices without the need for a physical body cavity search or the need for an x-ray.

Yet another advantage provided by an embodiment of the present invention is the provision of a surgical device accounting network. The network allows for a centralized accounting of surgical devices utilized within multiple hospitals and operating rooms therein.

The present invention itself, together with attendant advantages, will be best understood by reference to the following detailed description, taken in conjunction with the accompanying figures.

### BRIEF DESCRIPTION OF THE DRAWING

For a more complete understanding of this invention reference should now be had to the embodiments illustrated in greater detail in the accompanying figures and described below by way of examples of the invention wherein:

FIG. 1 is a block diagrammatic view of a smart fluid absorbing device for use during an operating procedure in accordance with an embodiment of the present invention;

FIG. 2A is a block diagrammatic view of a smart fluid absorbing device detection and accounting system utilizing serially coupled transponder antennas in accordance with an embodiment of the present invention;

FIG. 2B is a block diagrammatic view of a smart fluid absorbing device detection and accounting system utilizing wireless transponder antennas in accordance with an embodiment of the present invention;

FIG. 3 is a block diagrammatic view of surgical fluid absorbing apparatus accounting network in accordance with an embodiment of the present invention;

FIG. 4 is a logic flow diagram illustrating a method of accounting for surgical fluid absorbing apparatuses used during an operating procedure in accordance with an embodiment of the present invention;

FIG. 5 is a block diagrammatic view illustrating transitions of smart surgical fluid absorbing apparatuses before use during an operating procedure in accordance with an embodiment of the present invention; and

FIG. 6 is a logic flow diagram illustrating a method of detecting surgical fluid absorbing apparatuses within a body cavity in accordance with an embodiment of the present invention.

### DETAILED DESCRIPTION

In the following figures, the same reference numerals will be used to refer to the same components. While the present invention is described with respect to a system and method of detecting and accounting for surgical apparatuses used during an operating procedure, the present invention may be adapted to be used in other similar applications where such accounting is desired.

In the following description, various operating parameters and components are described for one constructed embodiment. These specific parameters and components are included as examples and are not meant to be limiting.

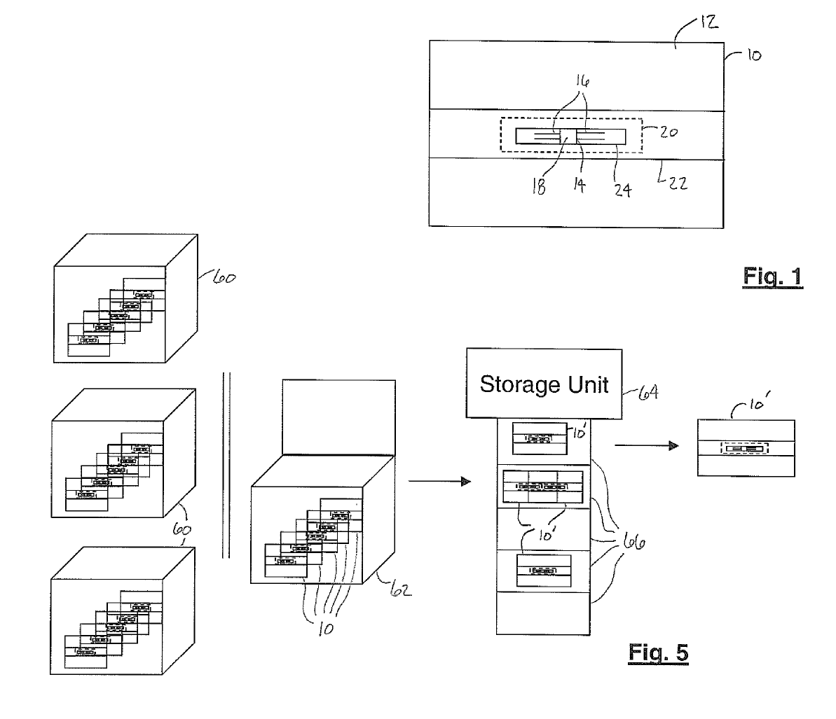

Although FIGS. 1-6 are primarily described with respect to fluid-absorbing apparatuses, the present invention may be applied to any surgical apparatus that may be left within a body cavity during an operating procedure. A surgical apparatus may for example be an operating instrument, a utensil, a tray, a gurney, or some other surgical apparatus known in the art.

Referring now to FIG. 1, a block diagrammatic view of a smart fluid absorbing device 10 for use during an operating procedure is shown in accordance with an embodiment of the present invention. The smart device 10 includes a surgical fluid absorbing apparatus 12 and a smart control circuit 14. The smart circuit 14 is attached to the absorbing apparatus 12 and stores identification information. The smart circuit 14 may be located anywhere on or embedded anywhere in the absorbing apparatus 12. As implied above although the smart circuit 14 is applied to the absorbing apparatus 12, it may be applied to any surgical apparatus known in the art.

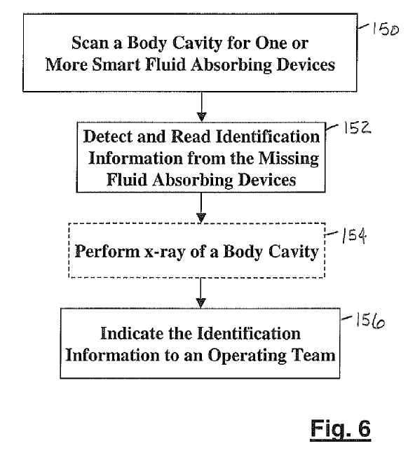

The absorbing apparatus 12 may be of various types, styles, sizes, and shapes. The absorbing apparatus 12 may be in the form of a cloth, a sponge, a towel, a pad, a swab, or other fluid absorbing apparatus known in the art. The absorbing apparatus 12 may be formed of various materials, such as cotton, gel, foam, thermoplastic, synthetic resin, natural rubber, synthetic rubber, cellulose, and nylon, or may be formed of some other material or material composite known in the art. The absorbing apparatus 12 may be packaged in groups, such that more than one fluid absorbing apparatus is contained within a single package. In one embodiment of the present invention, the fluid absorbing apparatuses are packaged in groups of five, as is shown in FIG. 5 and as described further below.

The smart control circuit 14 includes a transponder 16 and a logic circuit 18. The transponder 16 and the logic circuit 18 may be encased within a hypoallergenic casing, as is designated by dashed lines 20. The casing 20 is attached to a radio opaque marker or strip 22. The transponder 16 allows for communication transfer of the identification information stored in the logic circuit 14. The hypoallergenic casing 20 is used to prevent allergic reactions. During production of the smart device 10 the logic circuit 14 and the transponder 16 are mounted on the backing board 24, encased in the casing 20, and attached to the strip 22.

The transponder 16 may be in the form of an antenna and used to receive inductively generated identification signals containing identification information. The identification information may include an identification number or code that is designated for that particular smart device. Before use of the smart device 10 in an operating procedure, the identification information may be stored on the logic circuit 14 and periodically scanned to perform an accounting of all fluid absorbing devices used during that procedure. The term “accounting” refers to the difference between the number of fluid absorbing devices actually in use and the number of fluid absorbing devices accounted for, which includes the fluid absorbing devices that are registered for use, are in use, and have been removed and/or discarded after use.

The logic circuit 14 may be a solid-state silicon based circuit or may be in some other form known in the art. The logic circuit 14 may be read or written to using radio frequencies. The use of radio frequencies to communicate identification information regarding smart fluid absorbing devices may be referred to as radio frequency identification (RFID) or inductive transponder technology.

The backing 24 may be formed of a high temperature resilient material such that it is capable of withstanding temperatures required for sterilization. Sterilization temperatures for fluid absorbing devices can exceed 105° C.

Although a hypoallergenic casing is utilized, other casings may be used. The hypoallergenic casing 20 may be attached to the strip 22 via an adhesive, may be stitched to the fluid absorbing apparatus 12 or the strip 22, or may be attached using some other attachment technique known in the art.

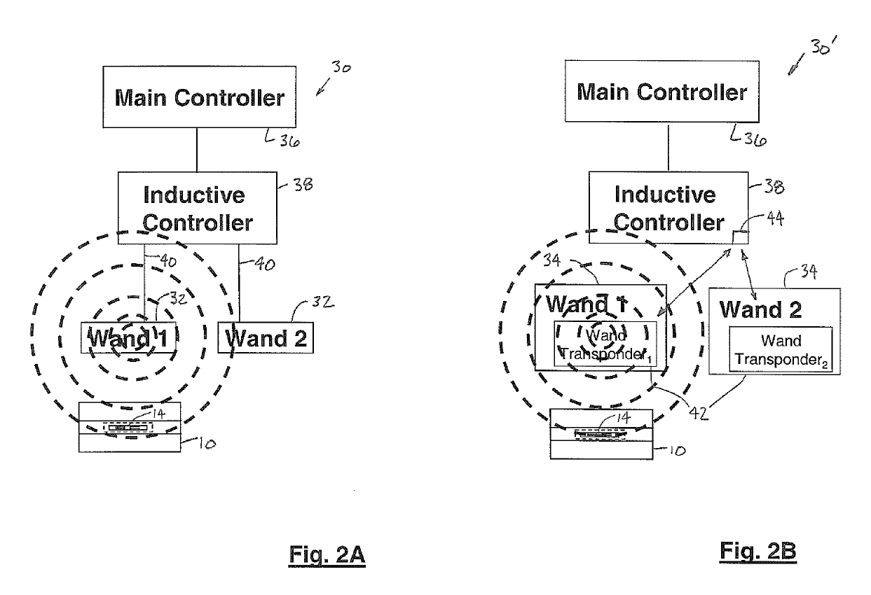

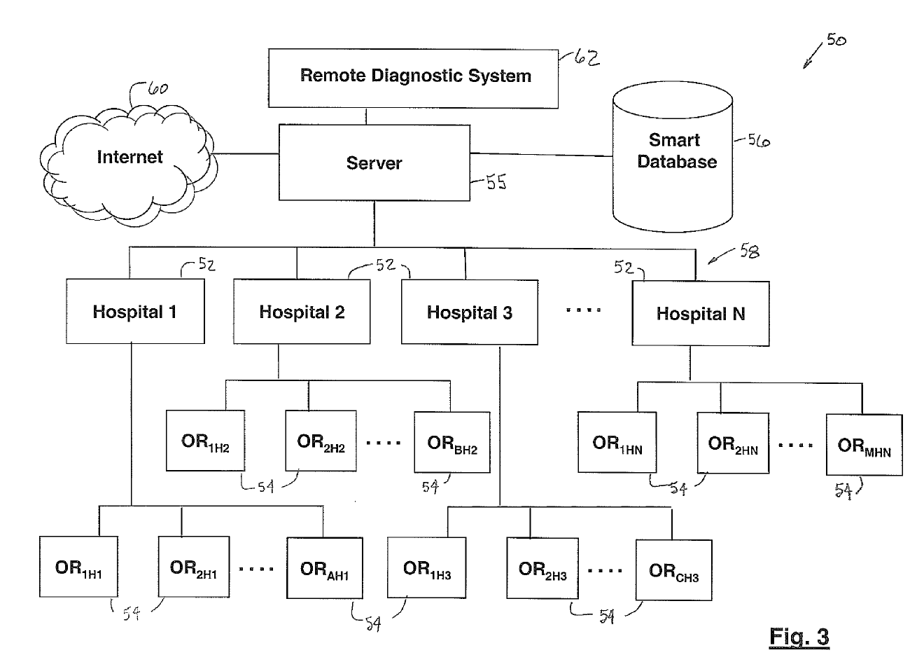

Referring now to FIGS. 2A and 2B, block diagrammatic views of smart fluid absorbing device detection and accounting systems 30 and 30′ are shown utilizing serially coupled transponder antennas 32 and wireless transponder antennas 34, respectively, in accordance with an embodiment of the present invention. The accounting systems 30 and 30′ include main controllers 36, inductive controllers 38, and one or more transponder antennas or wands, such as antennas 32 and 34. The main controllers 36 contain an application software, such as for example the smartCLOTH™ application software. Through the use of inductive transponder technology each smart surgical fluid absorbing device 10 is registered prior to use and is rapidly accounted for after an operation through a deregistration process.

The wands 32 and 34 may be in the form of mobile handheld devices or may be in the form of stationary scanning devices. The serial coupled wands 32 are electrically coupled to the associated inductive controller 38 via serial connections 40. Each of the wireless wands 34 contains a wand transponder 42 that is in wireless communication with an inductive controller transponder 44 of the associated inductive controller 38.

The main controllers 36 and the inductive controllers 38 may be desktop or laptop configured or may also be in the form of handheld devices. In one embodiment of the present invention, the main controllers 36 are desktop configured and the inductive controllers 38 are handheld configured.

The main controllers 36 and the inductive controllers 38 may be microprocessor based such as a computer having a central processing unit, memory (RAM and/or ROM), and associated input and output buses. The main controllers 36 and the inductive controllers 38 may be in the form of application-specific integrated circuits or may be formed of other logic devices known in the art. The main controllers 36 and the inductive controllers 38 may be a portion of a central main control unit, may be combined into a single controller, or may be stand-alone controllers as shown.

The wands 32 and 34 transmit and receive the identification information to and from the logic circuits 14 located on the absorbing devices 10. The wireless configuration of the wands 34 simplifies the registration and deregistration process of the absorbing devices 10 by removal of the serial connections 40.

The wands 32 and 34 and the inductive controllers 38 may be in the form of low-power non-disruptive inductive devices that transmit and receive short messages. As such, the wands 32 and 34 and inductive controllers 38 do not affect a body of interest being acted on and also do not affect equipment utilized in the operating procedure. Since the accounting systems 30 and 30′ are inductive no power sources or toxic materials are utilized inside the body to enable communications between the wands 32 and 34 and the control circuits 14.

Although radio frequency identification with inductance is utilized, the accounting systems 30 and 30′ may be modified to support long-range detection for activation, registration, and deregistration of absorbing devices.

Referring now to FIG. 3, a block diagrammatic view of surgical fluid absorbing apparatus accounting network 50 in accordance with an embodiment of the present invention is shown. Since many hospitals have several operating rooms, which may each be used to perform various operating procedures twenty-four hours a day, network communications may be utilized. The accounting network 50 thus includes multiple hospitals 52 having any number of operating rooms 54, which each have access to a smart database 56 via a server 55. The operating rooms 54 may access the database 56 via an Intranet 58 or an Internet 60. There are N number of hospitals 52 and each hospital has an associated number of operating rooms. For example hospital, has A number of operating rooms. There are M sets of operating rooms, each set corresponding to a particular hospital.

The server 55 and the smart database 56 may be centrally located and within one of the hospitals 52. The smart database 56 contains identification information related to the smart absorbing devices 10 utilized in each operating room 54, which may be archived and retrieved at a future date. The database 56 may store information such as surgery type, number of smart absorbing devices 10 accounted for and used, and any other pertinent information to safe guard the accountability of the smart absorbing devices 10. The database 56 may also store a proposed or recommended radio frequency identification technique to be used in a particular operation such as for surgical equipment or tools. The database 56 may be accessed by hospital administrators and may be access limited to a certain set of hospital administrators.

A remote diagnostic system 62 may be coupled to the server 55 and be used to ensure that the accounting network 50 and any accounting systems, such as systems 30 and 30′, function reliably. The remote diagnostic system 62 allows for remote administration of each hospital 52 and operating room 54. Remote administration provides a reliable real time surgical technique for management and accountability of smart absorbing devices. The diagnostic system 62 may also enable routine software upgrades and system performance enhancements. A technician may remotely access the accounting network 50 and determine the current status of the accounting network 50 and of any absorbing devices and accounting systems utilized therein. The remote diagnostic system 62 may be in direct communication with the server 55, as shown, or may be in communication via the Internet 60.

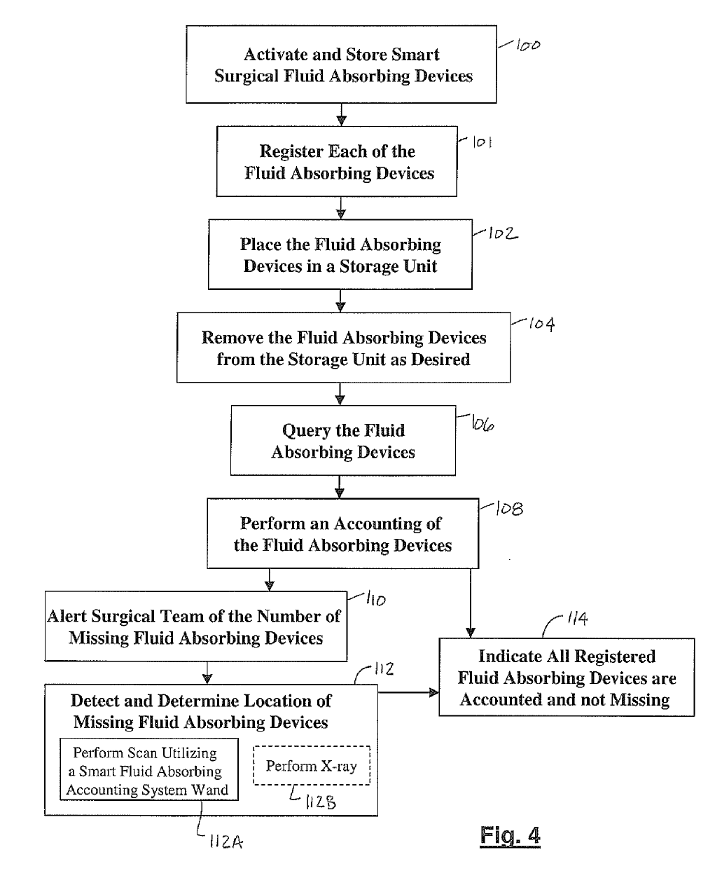

Referring now to FIGS. 4 and 5, a logic flow diagram illustrating a method of accounting for surgical fluid absorbing devices used during an operating procedure is shown along with a block diagrammatic view illustration of the transitions of the surgical fluid absorbing devices before use during the operating procedure.

In step 100, the smart fluid absorbing devices 10 to be stored and tentatively utilized during the operating procedure are activated. The absorbing devices may be activated via the wands 32 or 34. In FIG. 5, multiple packages 60 are shown, each of which having five smart fluid absorbing devices. Package 62 represents a package selected to be initialized.

In step 101, a smart fluid absorbing apparatus detection and accounting system, such as accounting systems 30 and 30′, is used to register each fluid absorbing device 10 prior to use during the operating procedure. A wand, such as one of the wands 32 or 34, is passed over the package 62 and/or each of the fluid absorbing devices 10. The fluid absorbing device package 62 may be passed over or near the wand when the wand is in a stationary configuration.

The main controller 36 or inductive controller 38 assigns a unique identification (ID) for each absorbing device 10 and programs or writes that identification to each of the logic circuits 14. The ID can include an operating room number, surgical lead, patient name, operation type, an absorbing device number, as well as other identification information. In a simple example embodiment, a serially increasing fluid absorbing device number may be used as the ID.

In step 102, the absorbing devices 10 are placed within a storage unit or bin 64. The absorbing devices 10′ within the bin are registered. The bin 64 may be compartmentalized, as shown. Each compartment 66 may correspond to a particular type of absorbing apparatus. In step 104, the absorbing devices 10′ are removed from the storage unit 64 as desired for use during the operating procedure.

In step 106, as the absorbing devices 10 are used and removed from the body they may be returned to the storage unit 64 and scanned or they may be scanned and then disposed. Each absorbing device 10 is queried and read after the operating procedure. The absorbing devices may be queried during the operating procedure when it is desirable to dispose of an absorbing apparatus. The same wand may be used to query the absorbing devices during and after the operating procedure as that used to register the absorbing devices before the procedure.

In step 108, the main controller 36 compares the number of absorbing devices 10 that were registered and placed within the storage device 64 before the procedure with that existing in the storage device 64 during or after the procedure to assure that none of the absorbing devices 10 are unaccounted for or remain in the body cavity. When there exists a discrepancy between the number of absorbing devices registered and the number of absorbing devices existing during or after the procedure, the main controller 36 indicates to an operator, administrator, or other individual the discrepancy. Query time using the above-described technique is significantly reduced over that of prior techniques. Reduced query time reduces costs involved therein.

In step 110, when one or more fluid absorbing devices are not detected in step 108, but has been assigned as “used” during registration, the main controller 36 alerts the surgical team performing the operating procedure of the missing devices and there identification information. The surgical team may then proceed to perform an extensive search of the body to locate the missing absorbing devices. The accounting system utilized may message, request, or alert hospital staff for assistance. This messaging may be in the form of a text message. An administrator may initiate the alert.

In step 112, the missing absorbing devices are detected and location thereof is determined. In step 112A, the wand is used to detect the missing absorbing devices. The wand may be used to isolate the absorbing device within the body. Once detected the absorbing devices are removed.

The accounting system utilized may be configured to scan the storage unit 64 autonomously, in the form of a deregistration. Autonomous deregistration increases efficiency of the accountability procedure. When the absorbing devices are not stored within a storage unit, a manual deregistration process may be utilized. At the end of the operating procedure the absorbing devices that were initially registered for the operating procedure are compared to those deregistered. It may be the case that not all absorbing devices registered are used. Consequently any absorbing devices not used in the operation are deregistered and disposed. The disposal satisfies sterilization procedures.

In step 112B, an x-ray of the body may also be performed to detect the missing absorbing devices as desired. The opaque strip of the absorbing devices may be detected through the use of an x-ray machine.

In step 114, when all of the absorbing devices registered are accounted for the main controller 36 indicates such to the operating team. Any missing absorbing device alerts are deactivated and operating room data is stored in a database, such as the database 56.

The alerts in steps 110 and 114 may be audio and/or video in nature. The alerts for a missing absorbing device may have an alarming sound and/or visual graphic warning indication. The alert that all absorbing devices registered are accounted for may have a pleasant sound and/or visual graphic indication.

Referring now to FIG. 6, a logic flow diagram illustrating a method of detecting at least one surgical fluid absorbing device within a body cavity is shown.

In step 150, the body cavity is scanned using as an example one of the above-described wands and accounting systems. In step 152, when an absorbing device is detected the accounting system reads the identification information stored on the control circuit of that absorbing device and generates an identification signal. In step 154, an x-ray of the body cavity may also be performed to further aid in the detection and location determination of the absorbing devices.

In step 156, an accounting system, such as one of the accounting systems 30 and 30′, generates an identification signal in response to the identification information and indicates to the operating team the detection of the absorbing device and any related identification information.

The above-described steps with respect to the methods of FIGS. 4 and 6 are meant to be illustrative examples; the steps may be performed sequentially, synchronously, simultaneously, or in a different order depending upon the application.

The present invention provides a smart surgical fluid absorbing device, accounting system, and network for the efficient detection and accounting of fluid absorbing devices utilized during an operating procedure. The system and methods of the present invention are simple and inexpensive in design.

While the invention has been described in connection with one or more embodiments, it is to be understood that the specific mechanisms and techniques which have been described are merely illustrative of the principles of the invention, numerous modifications may be made to the methods and apparatus described without departing from the spirit and scope of the invention as defined by the appended claims.

What is claimed is:

1) A smart surgical device for use during an operating procedure comprising:
a surgical apparatus for aiding in the facilitation of an operating procedure; and
a body internal readable control circuit coupled to said surgical apparatus and storing identification information related to said surgical apparatus.

2) A smart device as in claim 1 further comprising a transponder coupled to said body internal readable control circuit and receiving an identification signal, said body internal readable control circuit storing said identification information in response to said identification signal.

3) A smart device as in claim 2 wherein said transponder is in the form of an antenna.

4) A smart device as in claim 2 wherein said body internal readable control circuit stores said identification information in response to an inductive source.

5) A smart device as in claim 1 further comprising a mounting structure, said body internal readable control circuit coupled to said mounting structure.

6) A smart device as in claim 5 wherein said mounting structure is in a high temperature structure.

7) A smart device as in claim 1 further comprising a protective enclosure coupled to said surgical apparatus, said body internal readable control circuit contained within said protective enclosure.

8) A smart device as in claim 7 wherein said protective enclosure is hypoallergenic.

9) A smart device as in claim 7 wherein said protective enclosure is adhered to said surgical apparatus.

10) A smart device as in claim 7 wherein said protective enclosure is stitched to said surgical apparatus.

11) A smart device as in claim 1 further comprising a radio opaque marker coupled to said surgical apparatus.

12) A smart device as in claim 11 wherein said radio opaque marker is directly coupled to a protective enclosure, said protective enclosure encasing said body internal readable control circuit.

13) A smart device as in claim 1 wherein said surgical apparatus is selected from at least one of a fluid absorbing apparatus, a cloth, a towel, a pad, a swab, a sponge, an operating instrument, a tray, a utensil, and a gurney.

14) A smart device as in claim 13 wherein said surgical apparatus is formed of at least one material selected from cotton, gel, foam, thermoplastic, synthetic resin, natural rubber, synthetic rubber, cellulose, and nylon.

15) A surgical apparatus accounting system comprising at least one controller scanning at least one smart surgical device prior to use during an operating procedure and maintaining a database of surgical apparatus identification information, said at least one controller scanning said at least one smart surgical device after use thereof and performing an accounting of said at least one smart surgical device.

16) A system as in claim 15 wherein said at least one controller in scanning said at least one smart surgical device after use thereof scans said at least one smart surgical device after removal from a patient or from use in an operating room.

17) A system as in claim 15 wherein said at least one controller in scanning said at least one surgical apparatus after use thereof scans said at least one smart surgical device after removal from a patient and from use in an operating room.

18) A system as in claim 15 wherein said at least one smart surgical device comprises:
at least one surgical apparatus;
a transponder for receiving an inductive identification signal; and
a control circuit coupled to said at least one surgical apparatus and said transponder, said control circuit storing identification information in response to said inductive identification signal.

19) A system as in claim 18 wherein said at least one controller performs said accounting in response to said identification information.

20) A system as in claim 15 wherein said at least one controller is inductive.

21) A system as in claim 15 further comprising:
at least one surgical apparatus;
at least one transmitter coupled to said at least one controller and transmitting said inductive identification signal to said at least one surgical apparatus; and
a circuit coupled to said at least one surgical apparatus and storing identification information in response to said inductive identification signal.

22) A system as in claim 21 wherein said at least one transmitter is in the form of a wand.

23) A system as in claim 21 wherein said at least one transmitter is in wireless communication with said at least one controller.

24) A system as in claim 21 wherein said at least one transmitter is battery powered.

25) A system as in claim 15 further comprising a circuit coupled to at least one surgical apparatus and storing identification information, said at least one controller performing said accounting in response to said identification information.

26) A system as in claim 15 wherein said at least one controller comprises:
a computer; and
an inductive controller.

27) A system as in claim 15 further comprising at least one storage unit storing said at least one smart surgical device prior to use thereof, said controller scanning said at least one smart surgical device prior to placement in said storage unit.

28) A system as in claim 27 wherein said storage unit is compartmentalized.

29) A system as in claim 15 wherein said at least one smart surgical device is packaged in groups.

30) A smart surgical device accounting network comprising:
a database;
a server coupled to said database; and
a plurality of controllers coupled to said server and scanning a plurality of smart surgical devices prior to use in at least one operating procedure and storing smart surgical device identification information in said database via said server.

31) A network as in claim 30 wherein said plurality of controllers scan said plurality of smart surgical devices after removal thereof and perform an accounting of said plurality of smart surgical devices in response to said identification information.

32) A network as in claim 31 wherein said plurality of controllers scans said plurality of smart surgical devices after removal from at least one patient or from use in at least one operating room.

33) A network as in claim 31 wherein said plurality of controllers scans said plurality of smart surgical devices after removal from at least one patient and from use in at least one operating room.

34) A network as in claim 30 wherein said plurality of controllers are assigned to a plurality of operating rooms.

35) A network as in claim 30 wherein said plurality of controllers are assigned to a plurality of medical centers.

36) A network as in claim 30 wherein said plurality of controllers access said database via an Internet.

37) A network as in claim 30 wherein said plurality of controllers access said database via an Intranet.

38) A system for detecting at least one surgical apparatus within a body comprising:
at least one circuit coupled to the at least one surgical apparatus and storing identification information; and
a scanning device detecting said at least one surgical apparatus, scanning said at least one circuit, and generating an identification signal in response to said identification information.

39) A system as in claim 38 further comprising an indicator indicating the presence of said at least one surgical apparatus in response to said identification signal.

40) A system as in claim 38 further comprising a controller performing an accounting of said at least one surgical apparatus in response to said identification information.

41) A method of accounting for at least one surgical apparatus used during an operating procedure comprising:
scanning at least one surgical apparatus prior to use during an operating procedure and maintaining a surgical apparatus database; and
scanning said at least one surgical apparatus device after use thereof and performing an accounting of said at least one surgical apparatus based on said surgical apparatus database.

42) A method of detecting at least one surgical apparatus within a body cavity comprising:
scanning the body cavity for the at least one surgical apparatus; and
detecting and reading a circuit of the at least one surgical apparatus and generating an identification signal in response to identification information read from the at least one surgical apparatus.
